(19) United States
(12) Patent Application Publication
(10) Pub. No.: US 2024/0374307 A1
(43) Pub. Date: Nov. 14, 2024

(54) SYSTEMS AND METHODS FOR PERCUTANEOUS DIVISION OF FIBROUS STRUCTURES WITH VISUAL CONFIRMATION

(71) Applicant: PAVmed, Inc., New York, NY (US)

(72) Inventors: Lishan Aklog, Purchase, NY (US); Brian J. deGuzman, Paradise Valley, AZ (US); Richard Yazbeck, Norwell, MA (US); Jim Pelletier, Northampton, MA (US); Kevin Owen, Crozet, VA (US); Timothy L. Moulton, Newport, RI (US); Taylor Bensel, East Walpole, MA (US); Mark Doherty, Lynnfield, MA (US); Adam Dixon, Charlottesville, VA (US)

(21) Appl. No.: 18/692,917

(22) PCT Filed: Sep. 27, 2022
(86) PCT No.: PCT/US22/77071

§ 371 Date: Mar. 18, 2024

Related U.S. Application Data
(60) Provisional application No. 63/248,578, filed on Sep. 27, 2021.

(51) Int. Cl. A61B 18/14; A61B 8/12; A61B 17/32; A61B 18/00; A61M 25/10

(52) U.S. Cl. CPC A61B 18/1492; A61B 8/12; A61B 17/320036; A61B 2018/00232; A61M 25/1002

(57) ABSTRACT

A device for dividing a fibrous structure may include a handle having a proximal end. a distal end. and an imaging core extending therebetween. the imaging core configured to image tissue including the fibrous structure: an expandable member positioned near the distal end of the handle, the expandable member being transitionable between an inflated state and a deflated state, and the expandable member includes a cutting element arranged on a surface of the expandable member for weakening or cutting the fibrous structure resulting in its division: and a probe cover coupled to the handle. the probe cover including a covering extending in a proximal direction for draping over the handle to create a sterile barrier between the handle and the expandable member.

### CROSS-REFERENCE TO RELATED APPLICATION

This application claims priority to, and the benefit of, U.S. Provisional Application No. 63/248,578, filed Sep. 27, 2021, for all subject matter common to both applications. The disclosure of said provisional application is hereby incorporated by reference in its entirety.

### BACKGROUND

The body contains a variety of anatomic compartments with one or more fibrous walls. In certain pathologic situations, the structures within the compartment can be compressed either by swelling or inflammation of the structures or constriction by the compartment walls. For example, compression of blood vessels or nerves passing through the compartment can lead to poor blood flow or loss of neurologic (sensory or motor) function in the tissues within or beyond the compartment. Examples of such conditions include carpal tunnel syndrome, plantar fasciitis, fascial compartment syndrome and abdominal compartment syndrome. The treatment of these conditions will often involve cutting one or more fibrous walls to release pressure on the compartment's anatomic structures. This usually requires open surgery either with direct or endoscopic vision. Few if any percutaneous options exist for these conditions.

Carpal tunnel syndrome (CTS) is the most common cumulative trauma disorder (CTD's) which collectively account for over half of all occupational injuries. It exacts a major economic burden on society including billions in lost wages and productivity. The carpal tunnel is located in the wrist. It is bounded by the carpal bones posteriorly, laterally and medially, and by the transverse carpal ligament anteriorly. The flexor tendons and the median nerve pass through the carpal tunnel. Cumulative trauma leads to inflammation within the tunnel and can manifest itself clinically through its compressive effect on the median nerve resulting it motor and sensory dysfunction in the hand. The diagnosis is usually confirmed with nerve conduction tests. Traditional surgical approaches are effective but invasive and have to be performed in a surgical operating room. An incision is made in the palm or over the wrist. The transverse carpal ligament is surgically exposed and divided with scissors or a scalpel. Endoscopic approaches are less invasive but more technically challenging, have been associated with a higher complication rate and are more expensive. Endoscopic approaches still require a 1 cm surgical incision and some initial surgical dissection before the endoscope is passed into the carpal tunnel. One device attempts to use a transillumination to guide blind passage of a protected knife. Another device passes a saw-like cutting device into the carpal tunnel blindly.

It is therefore desirable to have a percutaneous approach to treat carpal tunnel syndrome that is less invasive than existing approaches, which allows for visualization of internal biological structures and that results in less trauma and quicker recovery times for the patient.

### SUMMARY

In some examples, a device for dividing a fibrous structure includes a handle having a proximal end, a distal end, and an imaging core extending therebetween, a probe cover couple-able to the handle, the probe cover including a covering for draping over the handle, an expandable member positioned near the distal end of the handle, the expandable member being transition-able between an inflated state and a deflated state, and a cutting element situated on an outer surface of the expandable member for weakening or cutting the fibrous structure resulting in its division.

In a non-limiting embodiment, a device for dividing a fibrous structure is disclosed. The device includes a handle having a proximal end, a distal end, and an imaging core extending therebetween, the imaging core configured to image tissue including the fibrous structure; an expandable member positioned near the distal end of the handle, the expandable member being transitionable between an inflated state and a deflated state, and the expandable member includes a cutting element arranged on a surface of the expandable member for weakening or cutting the fibrous structure resulting in its division; and a probe cover coupled to the handle, the probe cover including a covering extending in a proximal direction for draping over the handle to create a sterile barrier between the handle and the expandable member.

In some embodiments, the expandable member can be a balloon. The expandable member can have an elongated cross-sectional shape. The expandable member can be configured to contact the fibrous structure and expand outwards to tension the fibrous structure across the cutting element. The cutting element can be situated along a longitudinal dimension of the expandable member. The expandable member can expand radially so as to tension the fibrous structure in a direction substantially transverse to the cutting element. The cutting element can be configured to emit electrical or thermal energy to weaken or cut the fibrous structure.

In some embodiments, the imaging core can be translatable relative to the expandable member. The imaging core can be an ultrasound transducer. The imaging core can be a cylindrical ultrasound transducer configured to circumferentially image tissue. The imaging core can be translatable relative to the handle. In some embodiments, the device can further include a series of dilators capable of being coupled to the probe cover.

In some embodiments, the device can further include a tear-away sheath configured to be disposed over at least one of the series of dilators and to be removably coupled to the at least one of the series of dilators. The expandable member can be coupled to the probe cover. The device can further include a secondary expandable member disposed within a tear-away sheath, the secondary expandable member being capable of being coupled to the probe cover and configured and arranged to dilate tissue.

In a non-limiting embodiment, a method for dividing a fibrous structure is provided. The method includes successively introducing a series of dilators with increasing diameters through an incision into a tissue compartment that includes the fibrous structure; positioning, proximate the fibrous structure, a division device including an expandable member having a cutting element situated thereon; expanding the expandable member outwards to tension the fibrous structure across the cutting element; providing an imaging core into the tissue compartment; imaging the tissue compartment with the imagining core; and activating the cutting element to weaken or cut the fibrous structure while displaying an image from the imaging core in real-time.

In some embodiments, the method can further include introducing a tear-away sheath into the tissue compartment over at least one of the series of dilators and inserting the expandable member through the tear-away sheath. The method can further include removing the tear-away sheath from the tissue compartment prior to expanding the expandable member. The cutting element can include an electrocautery lead, and wherein the step of activating the cutting element can include delivering radiofrequency energy to the electrocautery lead. The step of providing an imaging core into the tissue compartment can further include providing an imaging core at least partly through the division device.

### DESCRIPTION OF THE DRAWINGS

FIG. 1 depicts a schematic view of an anatomical compartment of the human body;

FIG. 2 depicts a device for percutaneous division of fibrous structures, in accordance with an embodiment of the present disclosure;

FIGS. 3A and 3B depict side and cross-sectional views of a device for percutaneous division of fibrous structures in deflated and inflated states, in accordance with an embodiment of the present disclosure;

FIG. 4 depicts a schematic view of the device of FIGS. 3A and 3B in an inflated state for tensioning a fibrous wall of an anatomical compartment;

FIGS. 5A-5C depict electrical/thermal cutting elements of a device for percutaneous division of fibrous structures, in accordance with an embodiment of the present disclosure;

FIGS. 6A-D are schematic representations of another example of a device for percutaneous division of fibrous structure having ultrasound imaging, in accordance with an embodiment of the present disclosure;

FIG. 7 is a schematic representation of various components of a system for percutaneous division of fibrous structures;

FIGS. 8A-B are schematic illustrations of a handle and a module of one embodiment of a division device; and

FIGS. 9A-B are schematic representations of a model of using a division device, and corresponding ultrasound images during the procedure.

### DETAILED DESCRIPTION

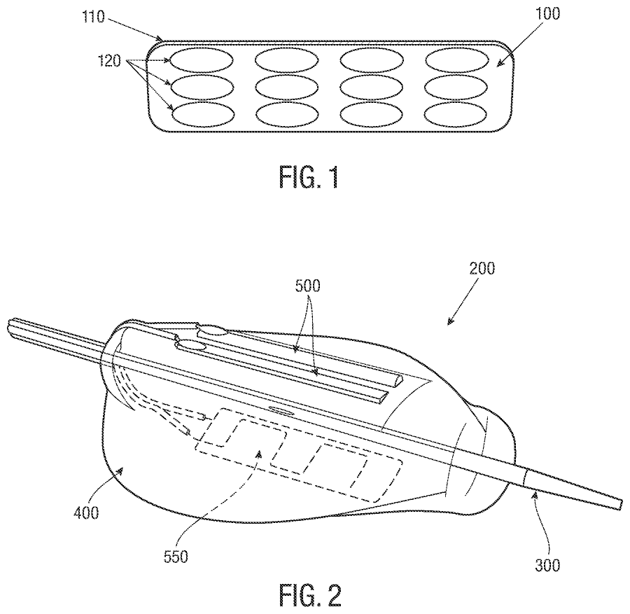

The present disclosure is directed to a medical device, and in particular, devices for percutaneous division of fibrous structures. While the devices and methods described herein may be used for percutaneous division of any sort of fibrous structure within the body, the present disclosure may, from time to time, refer to the treatment of carpal tunnel syndrome as an exemplary application. The carpal tunnel is an anatomic compartment in the wrist bounded by the carpal bones and the transverse carpal ligament. The clinical symptoms of carpal tunnel syndrome primarily arise from compression of the median nerve as it passes through the tunnel. Surgical division of the transverse carpal ligament relieves the compression of the median nerve and its associated symptoms. Referring to FIGS. 1 and 2, device 200, in various embodiments, may be utilized to divide a fibrous wall 110 of an anatomical compartment 100 within the body to relieve pressure on anatomical structures 120 within compartment 100.

Referring now to FIG. 2, percutaneous division device 200 of the present disclosure may generally include a catheter 300, an expandable member 400, one or more cutting elements 500, and one or more sensing/stimulating elements 550. Percutaneous division device 200 may be inserted into the body and advanced towards an anatomic compartment 100, such as the carpal tunnel, requiring treatment. Sensing/stimulating element 550 may optionally be utilized to help position device 200 within the compartment, and to avoid damaging any nearby nerves. Once properly positioned within the anatomic compartment, expandable member 400 may be expanded to apply a radial force generating lateral tension along a portion of the fibrous wall of the compartment. Cutting element 500 may be configured to engage the tensioned portion to divide the fibrous wall and thereby decompress the anatomic compartment for therapeutic effect.

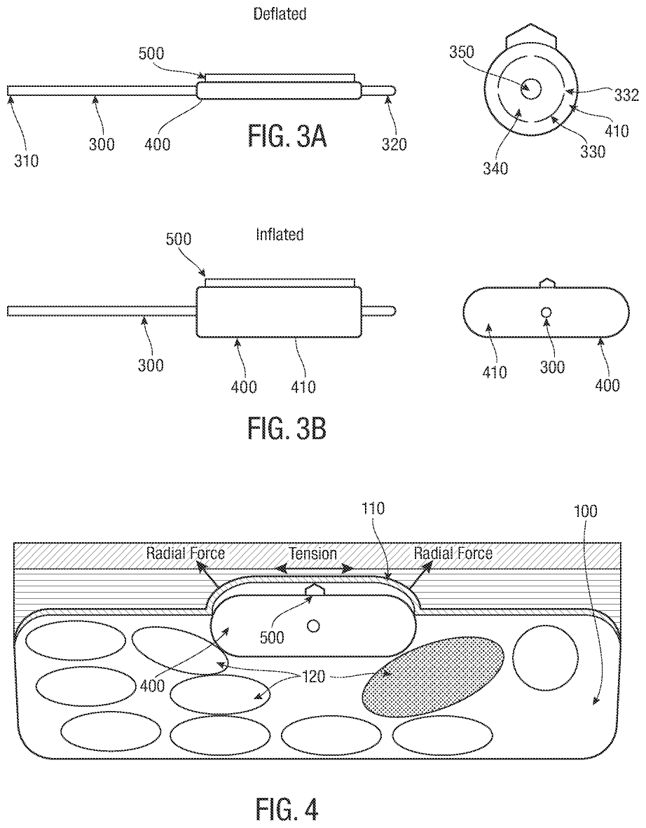

Referring now to the schematic views of FIGS. 3A and 3B, percutaneous division device 200 may include a catheter 300. Catheter 300, in various embodiments, may be rigid, semi-rigid or flexible. Catheter 300 may be made of any biocompatible material including plastic or metal. In embodiment, catheter 300 may be made of a flexible plastic material such as polyurethane, polyethylene or flourothermoplastic, among other suitable plastics.

Catheter 300, as shown, may have a proximal end 310, a distal end 320, and an outer surface 330. Catheter 300, in various embodiments, may include at least one lumen 340 through which fluids may be accommodated and directed between proximal end 310 and distal end 320. Catheter 300 may further include one or more openings 332 (shown in FIG. 3A as side holes) through which fluid may be directed between lumen 340 and an environment situated beyond outer surface 330 outside of catheter 300. Openings 332, in an embodiment, may be situated proximate distal end 320 so as to provide fluid communication between lumen 340 and an interior portion 410 of expandable member 400 positioned about a corresponding portion of outer surface 330 of catheter 300, as shown. In operation, fluid may be introduced into fluid lumen 340 at proximal end 310, directed towards distal end 320, and through openings 332 into interior portion 410 to inflate expandable member 400. Similarly, fluid may be withdrawn from expandable member 400 along the reverse path to deflate expandable member 400. Catheter 300, in various embodiments, may further include at least one lumen 350 for accommodating a guidewire 352 (not shown) for facilitating positioning of catheter 300 within compartment 100.

One of ordinary skill in the art will recognize that these are merely illustrative examples of suitable configurations of catheter 300, and that the present disclosure is not intended to be limited only to these illustrative embodiments.

Still referring to FIGS. 3A and 3B, percutaneous division device 200 may include expandable member 400, such as a balloon or similar expandable structure. For simplicity, expandable member 400 may be referred to herein as balloon 400 in the context of describing percutaneous division device 200; however, it should be recognized that expandable member 400 is not intended to be limited as such. Balloon 400, in an embodiment, may be substantially non-compliant, and can be made of a thin layer or a similar flexible plastic material.

Balloon 400 may be coupled to catheter 300 in a manner suitable for receiving and retaining fluid from lumen 340 of catheter 300 within interior portion 410 of balloon 400. In one such embodiment, balloon 400 may be positioned about a portion of outer surface 330 containing opening(s) 332 such that fluid directed through opening(s) 332 enters interior portion 410 of balloon 400. Balloon 400 may be bonded to catheter 300 to retain fluid directed into its interior portion 410 to allow for inflating the balloon 400 during the surgical procedure.

Referring now to FIG. 4, balloon 400 may be shaped to apply tension to fibrous wall 110. As balloon 400 is inflated, it pushes outward, generating a force in a radial direction on a portion of wall 110, which stretches that portion of wall 110 in a lateral direction. In various embodiments, cutting element 500 may be longitudinally oriented on balloon 400, meaning that the lateral tension created in wall 110 by balloon 400 acts in a direction substantially transverse to the longitudinally-oriented cutting element 500 situated on the surface of balloon 400. As configured, lateral tension causes wall 110 to become taut across cutting element 500, thereby making it easier to divide. In particular, as cutting element 500 weakens a contacted portion of wall 110, tension applied by balloon 400 facilitates division by pulling wall 110 apart along the weakened area. Further, as shown in FIG. 4, stretching wall 110 taut provides for wall 110 to be contacted by a discrete portion of cutting element 500 (e.g., the tip of cutting element 500, as shown), rather than with a wider portion cutting element 500 as may be the case if wall 110 were slack and allowed to conform around cutting element 500. Stated otherwise, the tension applied by balloon 400 allows cutting element 500 to act with high energy density on a small portion of wall 110, thereby providing for a cleaner cut with less tissue damage, which in turn may reduce the recovery period for the patient.

Still referring to FIG. 4, balloon 400 may be further shaped and sized to accommodate the specific anatomy of the compartment 100 within which it will be deployed. This may include, for example, being shaped and sized in a manner suitable for manipulating the position of, or minimizing pressure applied to, anatomical structures 120 situated within compartment 100. This may serve to protect these anatomical structures 120 from damage resulting from contact with cutting element 500 and/or to dissect tissues within the compartment to create more space for the anatomic structures within the compartment. As shown in FIG. 4, in an embodiment, balloon 400 may have an elongated cross-section (e.g., ovular) which, when positioned against fibrous wall 110, provides contact between an elongated side of balloon 400 and fibrous wall 110 that prevents anatomical structures 120 from sliding around its lateral ends and towards the site of division, where they could be damaged by cutting element 500. In another embodiment, balloon 400 may be provided with a substantially circular cross-section (not shown) with a large enough diameter sufficient to push nearby tendons, nerves, or other anatomical structures 120 outward from device 200 when inflated.

In some embodiments, balloon 400 may be provided with a variety of other cross sections. For example, balloon 400 may have, without limitation, a substantially circular, ovular, rectangular, or triangular cross sectional shape, to help achieve the desired effect on wall 110 and/or anatomical structures 120. Additionally, or alternatively, multiple balloons or shaped members may be positioned in relation to one another to help form the overall shape of balloon 400. For example, a “pontoon”-like configuration is possible wherein two smaller balloons are positioned on opposing sides of a larger central balloon to help form an overall ovular shape. Of course, one of ordinary skill in the art will recognize any number of additional configurations for this purpose within the scope of the present disclosure.

Similarly, balloon 400 may be adapted to minimize contact with (and applying resulting pressure on) certain surrounding anatomical structures 120 within compartment 100. For example, the small vertical dimension of the elongated cross-sectional design of FIG. 4 may serve to minimize pressure exerted on median nerve 122 situated below the site of division, whilst its longer horizontal cross-sectional dimension may still serve to apply tension to fibrous wall 110 and push tendons 124 aside. Embodiments of balloon 400 may be provided with suitable longitudinal profiles adapted for similar purposes.

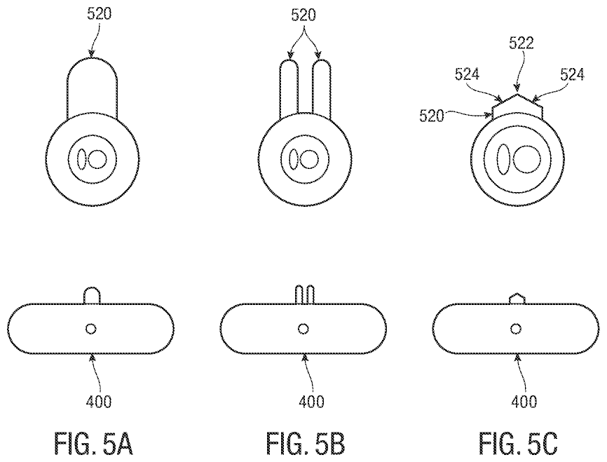

Referring now to FIGS. 5A-C, cutting element 500, in various other embodiments, may include an electrical element 520 configured to utilize electrical and/or thermal energy to divide fibrous wall 110. For example, cutting element 520 may include a unipolar or bipolar leads configured to communicate electrically with an electrocautery generator, as shown in FIGS. 5A and 5B, respectively. In operation, when balloon 400 is inflated and the electrocautery lead(s) is in contact with fibrous wall 110, the electrocautery generator may be activated to deliver radiofrequency energy to the electrocautery lead(s). The radiofrequency energy heats and cuts the contacted, tensioned portion of the fibrous wall 110 and the fibrous wall 110 is divided under the pressure of balloon 400, thereby relieving the pressure in compartment 100, as described in more detail later in the disclosure. A bipolar configuration may be used in anatomic areas with critical structures (nerves, blood vessels) in the vicinity, as it limits the thermal spread of the radiofrequency energy. Leads 520 attached to alternative energy sources, such as microwave and laser light, may also be applicable in certain applications.

Embodiments of cutting element 520 utilizing electrical and/or thermal energy for division, in an embodiment, may further have a sharp knife-like edge (not shown) so that fibrous wall 110 is divided using both electrical and mechanical means. Similarly, referring FIG. 5C, lead(s) 520 may be provided with a substantially triangular cross-section. As configured, the leading edge 522 of the triangularly-shaped lead 520 may serve to concentrate the electrical and mechanical, thereby providing highly-concentrated energy density along a fine line at the site of division. This may result in less tissue trauma, shorter cutting times, faster recovery times, and more precise division of the fibrous wall 110. Further, the sloping surfaces 524 of the triangularly-shaped lead 520 may serve to further spread (i.e., tension) the portion of fibrous wall 110 proximate leading edge 522, thereby further enhancing the ability of device 200 to cut and divide fibrous wall 110. Further, a portion of the surface of the lead 520 extending up sloping surfaces 254 may be coated with an insulating material, allowing further concentration of the energy density to leading edge 522.

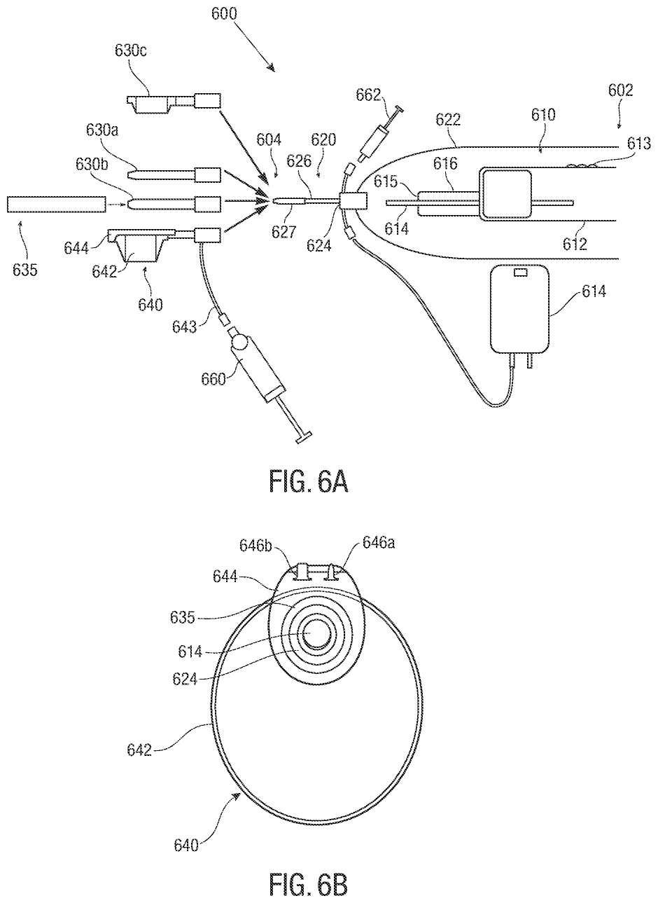

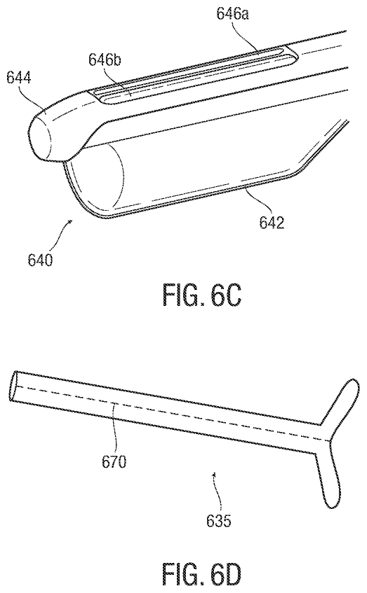

Turning now to FIG. 6A-6B, another embodiment of a system 600 for division of a fibrous tissue is shown. System 600 may generally extend between a proximal end 602 closer to the operator and distal end 604 disposed farther away from the operator. System 600 includes a handle 610 that includes a handheld body 612, a seating hub 616 that supports an inner ultrasound imaging core 614. In at least some examples, imaging core 614 may include a generally cylindrical ultrasound transducer configured and arranged to circumferentially image tissue and other device components disposed radially outward thereof. Imaging core 614 and seating hub 616 may be used to receive, accept, or otherwise support a plurality of components thereon.

In at least some examples, components of the system may be slid onto, or otherwise engaged with, imaging core 614 and/or hub 616 to mate therewith to complete various steps of the procedure. In some embodiments, the imaging core 614 and/or hub 616 can include retention features to allow those various components of the system to be locked, or fixed, relative to the imaging core 614 and/or hub 616. In some embodiments, the imaging core 614 and/or hub 616 can retain those various other components via an interference fit, without the need for other retention features. In at least some examples, hub 616 can include a sensor 615 (e.g., an RFID sensor or other similar sensor) capable of communicating with, and/or receiving data from, those components to recognize and/or identify the components that mate with the system 600. For example, using sensor 615 or other suitable techniques, system 600 may recognize or distinguish whether a balloon or a dilator are coupled to imaging core 614 and/or hub 616.

In some embodiments, the system 600 can include a probe cover 620. For example, the probe cover 620 can be mateable with the hub 616 by sliding the probe cover 620 onto the hub 616 and/or imaging core 614. In some examples, the probe cover 620 can click into place to confirm proper mating with the handle, e.g., via friction/interference fit. In some embodiments, the probe cover 620 can include a covering or bag-shaped drape 622 coupled to hub 624. The drape 622 in turn can be capable of being coupled to shaft 626. The drape 622 may be formed of latex, or other suitable material and may be configured and arranged to extend proximally from the hub 624 to cover the handle 610 and create a sterile barrier between the operator's hand and the cable, and the rest of the system that will be introduced into the patient's tissue. The probe cover 620, and specifically hub 624, may be releasably locked to handle 610. The robe cover 620 may include a gel-filled tip 627 and/or a gel overfill area to aid in an imaging process.

In some embodiments, the shaft 626 of probe cover 620 can include a radiofrequency contact window which can allow energy to pass therethrough. In some examples, shaft 626 may be formed of PEBAX® elastomer or other suitable material. The shaft 626 can have any suitable dimensions, for example, the shaft 626 can an outer diameter in the range of 2.0-6.0 mm, and in some cases the outer diameter is in the range of 3.7-4.0 mm. In some embodiments the shaft 626 can be used as a small dilator for performing the initial tunneling into the site of interest. Shaft 626 and hub 624 of probe cover 620 may, in turn, be configured to accept a number of other components.

For example, in some embodiments, a series of dilators 630a, 630b may be introduced over shaft 626 and configured to releasably mate and/or lock onto hub 624 of the probe cover. In some embodiments, the two dilators 630a, 630b can have different outer diameters, to slowly increase the diameter of the tunneling, to prevent injury to the patient. While two dilators 630a, 630b are shown, dilators 630a, 630b can include any number of serial dilators of different diameters that nest over shaft 626 and/or one another. For example, it will be understood that one, two, three, four, five or more dilators can used, as part of the system, during a procedure depending on the required insertion site and the tissue to be dilated. In at least some examples, dilator 630a can have a 6.0-6.5 mm outer diameter and dilator 630b can have an 8.5-9.0 mm outer diameter. Shaft 626 or dilator 630a may be used first to create the initial opening. The device 600 may then be removed from the patient, and the dilator 630a, 630b may be interchanged for the next larger dilator, which is used to progressively enlarge the opening.

In an additional, or alternative, dilation step, a tear-away sheath 635, as seen in FIGS. 6A-6D, can be disposed on, or over, the largest diameter dilator. In some embodiments, the sheath 635 can have a substantially cylindrical cross-sectional shape. It is contemplated that the sheath 635 can have a non-cylindrical cross-sectional geometry, including scaffolds with non-continuous walls or walls that include discontinuities. Sheath 635 may be left in place within the opening, such that the sheath 635 can be configured to maintain the shape and/or size of the patency in the tissue and/or to provide a smooth, atraumatic entryway for the introduction of a balloon or other treatment device. For example, a division device 640 can be disposed onto shaft 626 and optionally, locked to hub 624, and can be introduced through sheath 635 into the anatomy. In some embodiments, as seen in FIG. 6D, the sheath 635 can include scored, weakened portions 670 or other frangible sections along its surface, or on one side that allow it to tear away to form a single flat sheet or two or more portions and to remove it from the anatomy, while leaving the division device 640 in the appropriate position. Alternatively, in some embodiments, the sheath 635 may be removed without breaking or tearing it.

Alternatively, instead of using a series of dilators 630a, 630b, an expandable dilating member 630c may be used and the tear-away sheath 635 may be disposed over member 630c. In some examples, the expandable dilating member 630c may be in the form of an expandable balloon, or other expandable structures such as bellows. In an embodiment, the expandable dilating member 630c may be disposed over shaft 626 and an inflating device may be used to expand the diameter of member 630c (e.g., by introducing air, saline, or some other fluid into the interior of member 640c). Once inflated or expanded to the desired or predetermined size, member 630c may be removed from the patient, leaving behind sheath 635, and the final steps of the procedure may be carried out, including replacement of member 630c with division device 640 and the cutting of tissue.

In some embodiments, the division device 640 can include an expandable member 642 configured to apply a radial force generating lateral tension along a portion of the fibrous wall of a compartment. In at least some examples, expandable member 642 may include a balloon or similar expandable structure coupled to an inflation line 643 for receiving a fluid from a fluid source, such as an endoflator 660. The endoflator 660 may introduce and/or draw a fluid medium (e.g., water, saline, air, etc.) through inflation line 643 to expand and/or collapse member 642. In some embodiments, the endoflator 660 can be a syringe or other pressurized fluid source. The expandable member 642 may be substantially non-compliant and can be made of a thin layer or a similar flexible plastic material. In some embodiments, the expandable member 642 may be introduced through sheath 635 in a collapsed condition. As the expandable member 642 is inflated, it can push outward, generating a radially directed force, or pressure, on a portion of a tissue wall, which stretches that portion of wall in a lateral direction, similar to the expandable member 400 as show in FIG. 4 and discussed above.

In some embodiments, the system 600 can include a fluid introducer 662. The fluid introducer 662 can be in the form or a syringe, bag, or the like, and may be used to introduce a substance (e.g., saline, gel, etc.) into the nonsterile component of the device. For example, the fluid introducer 662 can introduce fluid through the hub 624. In some embodiments, the fluid introducer 662 can introduce saline, or other fluids, between imaging core 614 and probe cover 620. A second fluid introducer 664, again in the form of a syringe or bag, may be used to introduce saline, gel, or the like between other components (e.g., between probe cover 620 and the various dilators, or between probe cover 620 and division device 640 or expandable dilating member 630c, etc.).

In some embodiments, the expandable member 642 can include cutting elements 644 which may be longitudinally oriented on balloon 400, meaning that the lateral tension created in a wall by the expandable member acts in a direction substantially transverse to the longitudinally-oriented cutting element 644 situated on the surface of balloon. The expandable member 642 may be formed in any of the configurations, sizes, shapes, or orientations described above and may be configured to exert a force to keep tissue taut across a cutting element 644 for easier division.

Turning to FIGS. 6B and 6C, the cutting element 644 can include electrical and/or thermal energy elements to divide a fibrous wall. For example, the cutting element 644 may include a unipolar or bipolar leads configured to communicate electrically with an electrocautery generator, as previously described. In one example, the cutting element 644 may include a triangular active lead 646a and a flat return lead 646b. In a stimulating mode, either or both leads 646a, 646b can be used to deliver a stimulating signal to confirm that the nerve is not in the vicinity of the cutting element. In a cutting mode, the bipolar electrical energy may be delivered between the active lead 646a and the return lead 646b. In operation, when the expandable member 642 is inflated and the electrocautery leads 646a, 646b are in contact with fibrous wall 110, an electrocautery generator (not shown) may be activated to deliver radiofrequency energy to the electrocautery leads 646a, 646b. The radiofrequency energy can heat and cut the contacted, tensioned, portion of the fibrous wall 110 and the fibrous wall can be divided under the pressure of the expandable member 642, thereby relieving the pressure in a compartment.

In some examples, the system 600 can additionally include an imaging core 614. The imaging core 614 may be axially translatable relative to hub 616, expandable member 642, and/or cutting element 644. In some embodiments, the imaging core 614 can include button(s) or actuators 613 which can be disposed on handheld body 612 may be used to drive the imaging core 614. In some embodiments, with the expandable member 642 inflated, a physician can drive the imaging core 614 proximally and/or distally to assess the anatomy and ensure that it is safe to cut. After the cutting element 644 is used to divide the tissue, the physician may again drive the probe proximally and/or distally to ensure that the entire target (e.g., the transcarpal ligament) has been cut. Images can be saved to document the procedure.

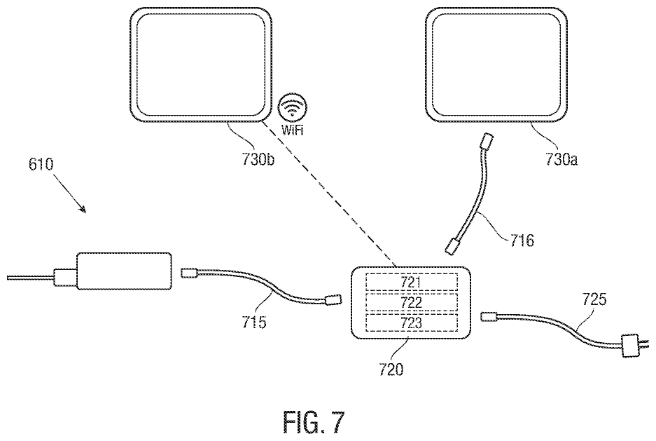

In some embodiments, as shown in FIG. 7, the handle 610 of system 600 can be coupled via cable 715 to a module 720 which can be used to display images from the imaging core 614. Module 720 may include a processor 721, a memory 722, and a transmitter 723. Module 720 may also be connected to a power source, such as an AC power source, via a cord 725. In some examples, the module 720 may be connectable via cabling 716 (e.g., HD-SDI or HDMI, 10 ft or 25 ft cables) to a first display 730a that can be disposed in the operating room or clinic. Alternatively, or in addition, module 720 may be wirelessly coupled to a second display 730b(e.g., an iPad or similar handheld device) via a peer-to-peer WiFi connection or other suitable wireless data transmission protocol, for example, via transmitter 723. In some examples, the second display 730b can also serve as a control panel and may be used for data entry (e.g., for receiving patient information via a keyboard, or through scanning a barcode via a camera). In some embodiments, the second display 730b can be used for capturing images, organization of information, entry of data into an EMR, and/or for using email and/or messaging capabilities to send the captured images from system 600. For example, the second display 730b can include a processor, a memory, CPU, and a transceiver. In some embodiments, the second display 730b may also be used as an additional display, for example, in learning settings, or as an alternative display where a primary display is unavailable. Thus, one or two display configurations are possible, and the two displays may present the same information (i.e., the two displays mirror one another), or present different information or images. In some embodiments, the system 600 can include more than two displays, as the system requires.

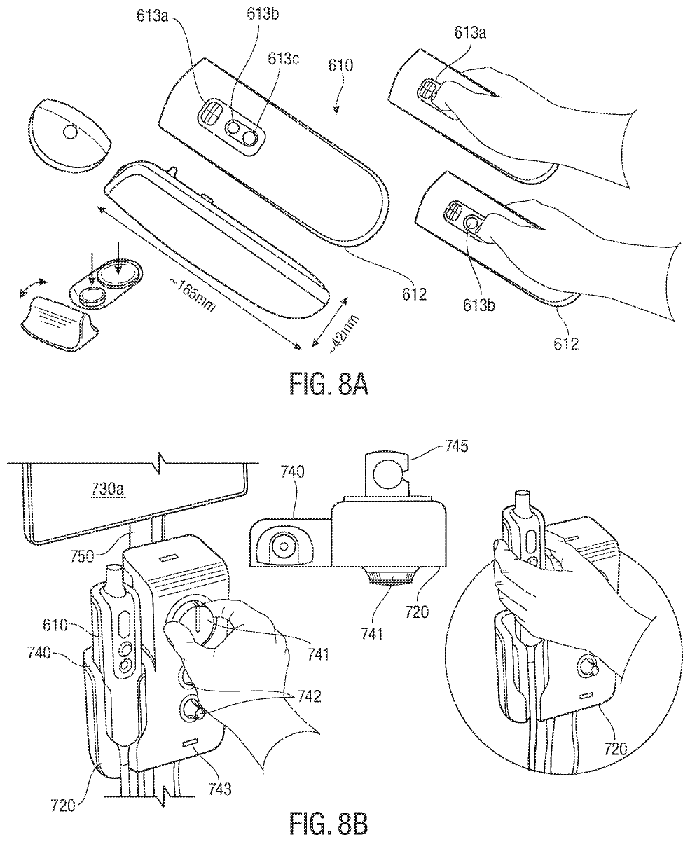

FIGS. 8A and 8B illustrate other embodiments of the system 600. In the embodiment shown in FIG. 8A, the system 600 can include a handle 610, or housing, which includes a handheld body 612 that can be grasped by the user's hand. In an embodiment, the handheld body 612 can include toggles or actuators to engage the system 600. For example, three toggles or actuators 613a, 613b, 613c can be disposed on a face, e.g., the top face, of the device to be manipulated, for example, by the user's thumb. Actuators 613a-c may include a series of elastomeric toggles or buttons, each corresponding to a function of system 600. For example, actuator 613a may be pushed forward or backward from a central, neutral, position to advance or retract the imaging core relative to the handle. A circular button 613b may be used to arm the device by turning on an electrocautery generator and a second button 613c may be pressed to deliver radiofrequency energy to the electrocautery lead(s).

In the embodiment shown in FIG. 8B, a system is shown including a housing, or module 720 having a holster 740 for receiving handle 610, and a bracket 745 for coupling to a pole 750. The bracket 754 can be a C-bracket that can be partially wrapped around the pole 750 and include an adjustment mechanism to tighten the bracket 754 about the pole 750. The holster 740 can be shaped and sized to retain the handle 610 such that the handle 610 is maintained within the module 720 in many, if not most, orientations for ease of access. The pole 750 may additionally support a display 730a and can include wheels or castors (not shown) at the lower end for easy transportation. The module 720 can include any number of different input means, for example dial 741 and buttons 742. The module 720 may include the dial 741 which can be actuated for setting the appropriate level of RF energy to be delivered. In some embodiments, the module 720 can include buttons 742 which can be for controlling characteristics of the display (e.g., gain, contrast, brightness, etc.). In some embodiments, the module 720 can include a USB receiver slot 743 to allow images to be sent to a USB or other external memory storage device for further storage and/or evaluation.

The various devices disclosed herein can be used in a variety of methods. For example, the method of using the device can include a method to divide the transverse carpal ligament. The method can be consistent with the general method. For example, the system can include ultrasound guidance which can be useful in positioning the device and ensuring that the ligament is properly divided. The ultrasound guidance can delineate the transverse carpal ligament and its association with the median nerve.

In one example, the forearm and hand can be sterilely prepped and draped with the hand in the hyperextended position. Local, regional, or general anesthesia may be instituted. A tourniquet may be used but may not necessary. Anatomic landmarks can be marked on the skin using palpation and ultrasound imaging of the wrist. The proximal and distal edges of the transverse carpal ligament can be identified as the path of the palmaris longus tendon. The path of the median nerve is followed as it passes into and out of the carpal tunnel deep to the transverse carpal ligament. Any anatomic anomalies (e.g., bifid median nerve) or other pathology can be identified. Measurements can be taken using ultrasound or other modalities including determining the width of the transverse carpal ligament. This allows the operator to select the appropriate size kit instruments and cutting balloon catheter.

In some embodiments, the skin entry site can be identified in the distal forearm several centimeters proximal to the proximal edge of the transverse carpal ligament. The entry site can be generally on the ulnar side of the parlmaris longus tendon and hence the median nerve providing a flat, straight trajectory to the proximal edge of the transverse carpal ligament. Alternatively, the skin entry point may be in the hand with the device passing through the carpal tunnel from distal to proximal. The device may also be designed to penetrate the carpal tunnel from a medial or lateral direction with the balloon inflating along the long axis of the tunnel although this approach introduces several additional challenges such as maneuvering around the radial and ulnar arteries.

In some embodiments, a needle may be inserted at the skin entry site and advanced from proximal to distal until it passes into the carpal tunnel just deep to the transverse carpal ligament. Internal, or external, ultrasound imaging can be used to confirm that the tip of needle enters the carpal tunnel in the correct location, on the ulnar side of median nerve. The needle may be used to inject fluid or local anesthetic into the carpal tunnel, if desired. This injection can be used to dissect tissues away from each other and create working space.

In some embodiments, a guidewire may be inserted into the needle and advanced through the carpal tunnel along a trajectory that runs just deep to the transverse carpal tunnel and, again, ulnar to the median nerve. The guidewire can generally have a straight tip and can be stiff enough that it can penetrate through the tissues bluntly. The tip of guidewire can be tracked by ultrasound as it passes through the carpal tunnel and exits past the distal edge of the transverse carpal ligament. The guidewire may pass a few centimeters past this edge to provide an adequate rail for the balloon catheter system 600. In some examples, a guidewire may be advanced further such that it exits through the skin of the palmar surface of the hand between the thenar and hypothenar eminences. The positioning of the guidewire can be performed under ultrasound guidance to assure that the guidewire exits cleanly and avoid critical hand structures such the arterial palmer arch. Once the tip of guidewire tents the skin of the hand, a small nick in the skin with a knife blade can allow it to exit. Alternatively, a needle can be advanced over the guidewire such that it penetrates the skin in the hand. The needle can then be removed.

An appropriately-sized dilator (e.g., the serial dilators 630a, 630b or a balloon-type dilating member 630c) is then selected and advanced through the incision. Using a balloon 630c or a series of progressively larger dilators 630a, 630b, an opening may be progressively enlarged to the appropriate size and sheath 635 may be left in place as the largest dilating element is removed. In some embodiments, the division device 640 can be introduced through sheath 635, and sheath 635 may be torn away and removed. The division device 640 can be positioned to ensure that its axial orientation is correct, with cutting element 644 positioned superficially, just under the transverse carpal ligament. The position of the division device 640 and the location of anatomical components may be studied via the imaging core 614. If desired, the user may translate the imaging core 614 relative to the handle 610 to examine a different location. The longitudinal position of the cutting element 644 can additionally be adjusted based on this examination so that cutting element 644 spans the entire width of the ligament. This positioning can be confirmed using ultrasound guidance from within the compartment. In some cases, the imaging core 614 can include visual and ultrasound sensors for multiple means of determining the position of the device. When the division device 640 is properly positioned, based on visual and/or

ultrasound confirmation, the expandable member 642 may be inflated to a specified pressure with fluid, such that the expandable member 642 has the desired dimension. The fluid can be any liquid including saline or contrast material including echo contrast material or gas including air, carbon dioxide or oxygen. Inflation of the expandable member 642 can be monitored by direct inspection and palpation of the hand or by ultrasound guidance. Using ultrasound guidance, the operator can confirm that the expandable member 642 inflates uniformly while maintaining its axial orientation and dissecting the transverse carpal ligament from the deeper structures including the median nerve. The position of division device 640 relative to the transverse carpal ligament may be assessed in real-time by monitoring the ultrasound images on the display(s).

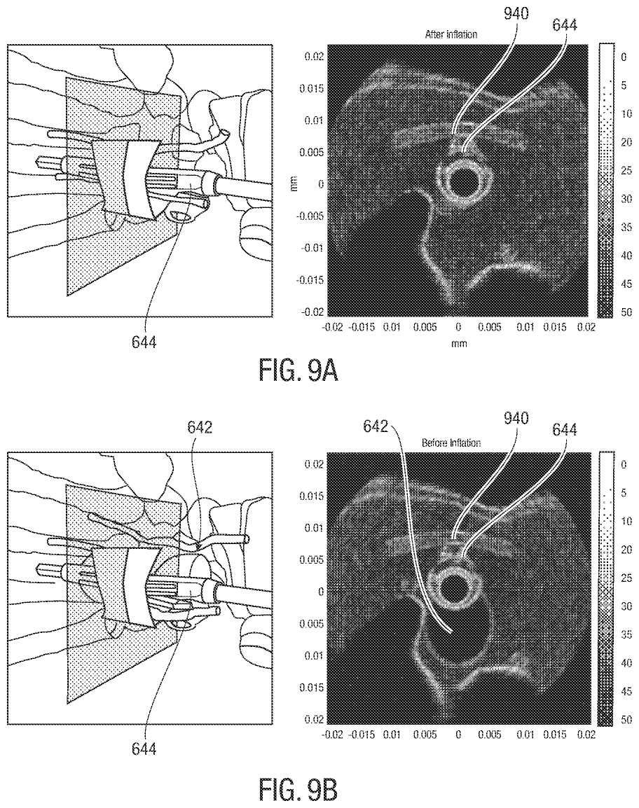

FIGS. 9A-B illustrate a model showing the use of a system 600 and a corresponding image on the display, e.g., displays 730a, 730b. As illustrated in FIG. 9A, a division device 640 of system 600 can be introduced through an incision in the distal forearm several centimeters proximal to the proximal edge of the transverse carpal ligament. In such a model, the division device 640 can includes expandable member 642 in the collapsed condition. Cutting element 644 including the leads can be disposed adjacent the transverse carpal ligament 940. In FIG. 9A, the transverse carpal ligament 940 is shown as a narrow band disposed above the two leads of cutting element 644 in the ultrasound images. If the position of division device 640 is not optimal, the expandable member 642 can be deflated and the device 600 repositioned before reinflating. For example, FIG. 9B illustrates the division device 640 properly oriented relative to the transverse carpal ligament 940 with the expandable member 642 being inflated so that the cutting element 644 presses on the ligament, which is made taut through that external force. As shown in FIG. 9B, the inflated expandable member 642 can appear as a large substantially oval or circular void on a side of the division device 600 opposite the cutting element 644.

Once the position of cutting element 644, relative to the transverse carpal ligament 940 and median nerve, is confirmed, the cutting element 644 can be activated. In some embodiments the positions of the cutting element 644 can be confirmed via the imaging core 614 and/or ultrasound. If the cutting element 644 is an electrocautery lead, it can be connected to a radiofrequency generator. The generator can be activated to deliver radiofrequency energy to the lead(s) 646a, 646b as it cuts through the ligament. The cutting process can be monitored in real-time via ultrasound on the display(s). In some embodiments the ultrasound can be disposed outside the patient, and alternatively, the ultrasound can be part of the imaging core 614. In some examples, edge detection software can be loaded onto the system 600 such that the software can utilize edge detection technology to automatically identify and/or label certain landmarks (e.g., the transverse carpal ligament). For example, the system may utilize software to identify points on the field of view where image brightness is sharply transformed past a certain predetermined threshold, or has discontinuities in the brightness, as seen in FIGS. 9A and 9B. Based on the discontinuities, a landmark (e.g., a ligament) may be identified, highlighted and/or shaded on the display so that they user can more easily locate the division device and its position relative to the landmark and/or confirm that the procedure is progressing as desired (e.g., by showing that the ligament is being divided properly).

Once the cutting process is completed, the expandable member 642 can be deflated and the completeness of the division of the transverse carpal ligament can be confirmed by the imaging core. The division device 640 and the guidewire can be removed and additional local anesthesia can be infiltrated into the wrist. Sterile dressings can be applied and appropriate post-operative care is instituted. The captured ultrasound images may be stored on the module 720, the display (e.g., iPad), an external storage device, a cloud-based service, or sent via email or text messaging capability to another physician or medical record. In some examples, the handle 610 and the imaging core 614 may be reusable, while probe cover 620, the dilators 630a-c, sheath 635 and the division device may be discarded.

While the present disclosure has been described with reference to certain embodiments thereof, it should be understood by those skilled in the art that various changes may be made and equivalents may be substituted without departing from the true spirit and scope of the disclosure. In addition, many modifications may be made to adapt to a particular situation, indication, material and composition of matter, process step or steps, without departing from the spirit and scope of the present disclosure. All such modifications are intended to be within the scope of the claims appended hereto.

What is claimed is:

1. A device for dividing a fibrous structure, the device comprising:
a handle having a proximal end, a distal end, and an imaging core extending therebetween, the imaging core configured to image tissue including the fibrous structure;
an expandable member positioned near the distal end of the handle, the expandable member being transitionable between an inflated state and a deflated state, and the expandable member includes a cutting element arranged on a surface of the expandable member for weakening or cutting the fibrous structure resulting in its division; and
a probe cover coupled to the handle, the probe cover including a covering extending in a proximal direction for draping over the handle to create a sterile barrier between the handle and the expandable member.

2. The device as set forth in claim 1, wherein the expandable member is a balloon.

3. The device as set forth in claim 1, wherein the expandable member has an elongated cross-sectional shape.

4. The device as set forth in claim 1, wherein the expandable member is configured to contact the fibrous structure and expand outwards to tension the fibrous structure across the cutting element.

5. The device as set forth in claim 4, where in the cutting element is situated along a longitudinal dimension of the expandable member.

6. The device as set forth in claim 5, wherein the expandable member expands radially so as to tension the fibrous structure in a direction substantially transverse to the cutting element.

7. The device as set forth in claim 1, wherein the cutting element is configured to emit electrical or thermal energy to weaken or cut the fibrous structure.

8. The device as set forth in claim 1, wherein the imaging core is translatable relative to the expandable member.

9. The device as set forth in claim 1, wherein the imaging core is an ultrasound transducer.

10. The device as set forth in claim 9, wherein the imaging core is a cylindrical ultrasound transducer configured to circumferentially image tissue.

11. The device as set forth in claim 1, further comprising a series of dilators capable of being coupled to the probe cover.

12. The device as set forth in claim 11, further comprising a tear-away sheath configured to be disposed over at least one of the series of dilators and to be removably coupled to the at least one of the series of dilators.

13. The device as set forth in claim 1, wherein the expandable member is coupled to the probe cover.

14. The device as set forth in claim 1, further comprising a secondary expandable member disposed within a tear-away sheath, the secondary expandable member being capable of being coupled to the probe cover and configured and arranged to dilate tissue.

15. The device as set forth in claim 1, wherein the imaging core is translatable relative to the handle.

16. A method for dividing a fibrous structure, the method comprising:
successively introducing a series of dilators with increasing diameters through an incision into a tissue compartment that includes the fibrous structure;
positioning, proximate the fibrous structure, a division device including an expandable member having a cutting element situated thereon;
expanding the expandable member outwards to tension the fibrous structure across the cutting element;
providing an imaging core into the tissue compartment;
imaging the tissue compartment with the imagining core; and
activating the cutting element to weaken or cut the fibrous structure while displaying an image from the imaging core in real-time.

17. The method for dividing a fibrous structure as set forth in claim 16, the method further comprising,
introducing a tear-away sheath into the tissue compartment over at least one of the series of dilators and inserting the expandable member through the tear-away sheath.

18. The method for dividing a fibrous structure as set forth in claim 17, the method further comprising,
removing the tear-away sheath from the tissue compartment prior to expanding the expandable member.

19. The method for dividing a fibrous structure as set forth in claim 16,
wherein the cutting element includes an electrocautery lead, and
wherein the step of activating the cutting element includes delivering radiofrequency energy to the electrocautery lead.

20. The method for dividing a fibrous structure as set forth in claim 16, wherein the step of providing an imaging core into the tissue compartment further includes providing an imaging core at least partly through the division device.
